(19) United States
(12) Patent Application Publication
(10) Pub. No.: US 2010/0057127 A1
(43) Pub. Date: Mar. 4, 2010

(54) Expandable Laminoplasty Fixation System

(76) Inventors: Brian McGuire, Carlsbad, CA (US); Sohaib Kureshi, Solana Beach, CA (US)

Correspondence Address: G. L. LOOMIS & ASSOCIATES, INC., 990 HIGHLAND DRIVE, SUITE 212Q, SOLANA BEACH, CA, 92075, US

(21) Appl. No.: 12/548,412

(22) Filed: Aug. 26, 2009

Related U.S. Application Data
(60) Provisional application No. 61/091,944, filed on Aug. 26, 2008.

(51) Int. Cl. A61B 17/70; A61B 17/80

(52) U.S. Cl. 606/246; 606/70

(57) ABSTRACT

The present invention relates to a medical implant for bone surgery, and more specifically to a vertebral fixation system with an expandable configuration. Such implants are particularly useful for securing and expanding transected spinal vertebrae following laminoplasty procedures.

### RELATED APPLICATION DATA

This application claims priority under 35 U.S.C. §119(e) to U.S. Provisional Application Ser. No. 61/091,944, filed Aug. 26, 2008.

### FIELD OF THE INVENTION

The present invention relates to a medical implant for bone surgery, and more specifically to a vertebral fixation system with an expandable configuration.

### BACKGROUND

In the spine the spinal cord and nerve roots are surrounded and protected by the vertebrae, which define an opening called the spinal canal through which the spinal cord passes. Ligaments and blood vessels are also present in the spinal canal. Spinal stenosis is a condition where there is narrowing of the spinal canal and often the neural foramen that causes compression of the spinal cord and/or nerve roots. Such a narrowing can be caused by numerous factors including bone spurs, degeneration of the intervertebral disks and facet joints, and thickening of the ligaments. Among the symptoms spinal stenosis can produce are pain and/or numbness in the arms, clumsiness of the hands, and gait disturbances.

Two surgical methods currently exist to create additional room in the spinal canal to decompress the spinal cord. The first is called a laminectomy, which involves removal of the lamina (roof) of one or more vertebrae. A limitation of the laminectomy procedure is that it involves removal of the supporting structures at the back of the vertebrae, which align the spinal column. The result may be that a patient suffers some postural deformity. To prevent such postural problems, a graft may be installed between the ends of the removed bone to span the void and reinstate the necessary support.

The second procedure is called a laminoplasty, in which the targeted vertebra is cut, spread apart and a laminoplasty plate is attached to bridge the hinged opening in the lamina. Normally a plate of an appropriate size is selected and bent to the desired shape and is fastened to the vertebra utilizing a plurality of screw holes. A strut of bone allograft can be inserted to permanently enlarge the space. Unlike the laminectomy, typically no structural bone material is excised during the laminoplasty procedure.

Two different laminoplasty procedures are in current use. The first is called the unilateral or “open door” laminoplasty in which one side (lamina) of the vertebra is cut all the way through, while the other side of the vertebra is cut only half-way through to create a hinge. The vertebral element is then rotated about the hinge, and the graft is inserted into the opening, increasing the opening of the spinal canal. The second procedure is called the bilateral or “French door” laminoplasty in which the midline of the vertebra (spinous process) is cut all the way through, and the lamina are cut half way through, creating two hinges. The vertebral element is then opened at the bisected spinous process, and a graft inserted into the opening, again increasing the opening of the spinal canal. Such laminoplasty procedures relieve pressure on the spinal cord while maintaining the stabilizing effects of the posterior elements of the vertebrae.

During a cervical “open door laminoplasty”, an incision is made on the back of the neck and a groove is cut down one side of the cervical vertebrae, creating a hinge. The other sides of the vertebrae are cut all the way through. At this point the spinous process is removed to allow the lamina bone flap to be swung open. A laminoplasty plate is then screwed to a facet and to the hinged open lamina. A plate of an appropriate size is selected and bent to the desired shape and preferably has a plurality of screw holes. A strut or wedge of bone can be placed in the open portion within the lamina and the facet to help hold the open position of the lamina. At the end of the procedure, the door of the vertebrae closes, wherein the laminoplasty plate and bone wedges prevent it from closing completely and the spinal cord and the nerve roots rest comfortably behind the door. By relieving pressure on the spinal cord it is the goal of laminoplasty to stop the progression of damage to the spinal cord and allow for as much recovery of function as possible.

A notable problem with such a laminoplasty procedure is that prior to performing such a procedure, the surgeon must measure the vertebra to determine the dimensions of the plate necessary for implantation. A laminoplasty implant is needed that allows its length to be varied during implantation while simultaneously distracting (opening) the surgically created gap in the lamina. Additionally such an expandable plate allows its length to be varied without changing its overall shape or configuration, so that a plate need not be selected and intensively custom shaped and formed prior to each surgery.

Various laminoplasty implants are known in the art. For example, U.S. Pat. No. 5,980,572 to Kim et al. and U.S. Pat. No. 6,080,157 to Cathro et al. each describes fixed-size implant designed to stabilize the lamina after open door or double door laminoplasty procedures. However such devices must be custom selected and intensively shaped and formed prior to each surgery and adjustment of the gap or space formed in a lamina after implantation is not possible. Another limitation of these implants and associated techniques is that a single implant extends to all the laminoplasty levels and does not provide well for lamina fusion, thereby being susceptible to stress fatigue.

U.S. Pat. No. 6,635,087 to Angelucci et al. describes implants for use in unilateral and bilateral laminoplasty procedures, wherein an implant of fixed length is installed between the cut segments of a transected vertebra and wherein the spinal canal is expanded. The implant is essentially a plate having ends that fasten to opposing segments of the transected vertebra and also has an intermediate portion configured to receive and hold a portion of bone allograft material. While such a device does provide for a level of lamina fusion it does requires custom selection and shaping prior to each surgery and adjustment of the gap or space formed in a lamina after implantation is not possible.

U.S. Pat. No. 6,660,007 to Khanna also describes fixation devices of fixed length for stabilization and fusion of vertebral laminae after laminoplasty procedures. These devices consist of a plate contoured at each end and of a length, width and thickness specific for vertebrae of the cervical, thoracic or lumbar spine and also have an intermediate portion configured to receive and hold a portion of bone allograft material. While such a device does provide for a level of lamina fusion it does requires custom selection and shaping prior to each surgery and adjustment of the gap or space formed in a lamina after implantation is not possible.

U.S. Pat. No. 7,264,620 to Taylor relates to an implant having first and second bases configured for securing two first and second cut portions, respectively, of a transected vertebra; wherein a connecting member can be configured for associating the first and second bases at a pre-selected spacing from each other. However, with such a device the spacing must be chosen prior to fixedly attaching the first and second bases to the two first and second cut portions of the vertebral laminae and adjustment of the spacing after attachment is not possible.

Therefore, there exists a need for an implant for use in either the open door or double door laminoplasty procedures is that allows the size to be varied after fixation to the laminae without changing the overall shape or configuration.

Also, there exists a need for an implant use in either the open door or double door laminoplasty procedures wherein the implant not have to be custom selected and intensively shaped and formed prior to each surgery.

Furthermore, there exists a need for an implant or use in either the open door or double door laminoplasty procedures wherein the implant is configured such it can be used to expand the gap or opening in the transected lamina to a precisely desired distance after the implant has been fixedly attached.

The implant devices of the present invention address these and other needs.

### BRIEF DESCRIPTION OF THE DRAWINGS

FIG. 1 depicts an isometric view of a bone fixation device of the invention attached to a lamina in the final position following a laminoplasty procedure.

FIG. 2 depicts an isometric view of an expandable lamina fixation device in a relatively closed or contracted position along with device-expanding forceps.

FIG. 3 depicts an isometric view of expandable lamina fixation device in a relatively open or expanded position along with device-expanding forceps.

FIG. 4 depicts an isometric view of an expandable bone plate 50 in an expanded or open position.

FIG. 5A depicts an orthographic top view of an expandable bone plate assembly provided with a ratchet mechanism.

FIG. 5B depicts an orthographic side view of the expandable bone plate assembly of FIG. 5A.

FIG. 5C depicts an isometric view of the bone plate assembly of FIG. 5A.

FIG. 6A is an orthogonal top view of an embodiment of an expandable bone plate.

FIG. 6B an orthogonal cut sectional end view of the expandable bone plate of FIG. 6A.

FIG. 7A is an orthogonal top view of an embodiment of an expandable bone plate.

FIG. 7B an orthogonal cut sectional end view of the expandable bone plate of FIG. 7A.

FIG. 8A is an orthogonal top view of an embodiment of an expandable bone plate.

FIG. 8B is an orthogonal bottom view of an embodiment of an expandable bone plate of FIG. 8A.

FIG. 8C an orthogonal cut sectional end view of the expandable bone plate of FIGS. 8A and 8B.

### SUMMARY OF THE INVENTION

The present invention describes an expandable and distractible implant that is used for stabilization of a lamina after an open door or double door laminoplasty procedure. Such a device offers controlled opening of a space within the lamina along with integrated fixation. In a typical laminoplasty procedure a lamina is first completely transected near the junction of the lateral mass and then a hinge is created on the contralateral side by scoring approximately half the thickness of the lamina. The transected lamina is expanded to create a laminar gap, a bone allograft is inserted into the gap and a gap-bridging plate is fastened at each side of the gap to stabilize the lamina. In preferred embodiments of the present invention the plate is comprised of two separate components, namely an inner component and an outer component. The plate is configured such that the proximal end the inner component is disposed within the proximal end of the outer component such that the inner and outer components are slidably coupled thus allowing the overall length of the assembled plate to be conveniently adjusted and locked into the desired position. Additionally, such an embodiment is useful for conveniently distracting (expanding) the lamina after the lamina has been surgically transected. In a typical use the distal end of either the inner or outer component is fixedly attached to the lamina on one side of the transection and the distal end of the other component is fixedly attached to the lamina on the opposing side of the transection and the components are then slidably adjusted to a desired overall plate length while separating the lamina at the transection to created a open space or gap in the lamina. In certain preferred embodiments the distal end of each of the two plate components comprises one or more holes through which a bone screw can be inserted to fixedly attach the elements to the lamina. Also in certain preferred embodiments the expandable plate also comprises a means for locking the two components of the plate into the desired relative positions position. Such a locking means can be, but is not limited to, a setscrew type mechanism. The expandable laminoplasty plates of the present invention are useful in open door (unilateral), double door (bilateral) and extensive laminoplasty procedures. In certain preferred embodiments the plate assembly is provided with a ratchet mechanism that allows the plate to be expanded (lengthened) along the longitudinal axis while preventing the plate from returning to a shortened position.

A typical bone fixation device of the present for use in the he lamina of the spine after laminoplasty comprises: an elongated fixation plate having adjustable length comprising a first plate component having a distal end, a proximal end and a longitudinal axis extending there between; said first plate component being slidebly coupled to a second plate component having a distal end, a proximal end and a longitudinal axis extending there between; wherein the distal end of the first plate component is configured to be fixedly attachable to a lamina facet on one side of a transected lamina and wherein the distal end of the second plate component is configured to be fixedly attachable to a lamina facet on the side of the transected lamina opposite the side to which the distal end of the first component is fixedly attachable; wherein the first plate component and second plate component can be slidably adjusted to produce a desired overall fixation plate length while the distal ends of the first and second plate components are each fixedly attached to opposing facets of the transected lamina and the two plate components are the locked relative to one another in the desired positions; and a locking means to secure the position of the first plate component and second plate component relative to one another.

In certain preferred embodiments the proximal end of the first plate component is slidably coupled within a pocket in the proximal end of the second plate component.

In certain embodiments the distal end of the first plate component and the distal end of the second plate component each have one or more bone screw receiving holes configured to permit fixed attachment to lamina facets with standard bone screws. Such bone screw receiving holes can assume any suitable shape such as round or elongate slots.

In certain preferred embodiments the locking means is a setscrew while in other preferred embodiments locking means is a ratchet mechanism. In certain embodiments, wherein the proximal end of the first plate component is slidably coupled within a pocket in the proximal end of the second plate component, the locking means is a ratchet mechanism comprising a series of tooth-like protrusions set an angle less of than 90 degrees along a surface of a first plate component and skewed toward the opening of a second plate component pocket and wherein the second plate component comprises a prawl for engaging the tooth-like protrusions of the first plate component, wherein the tooth-like protrusions and the prawl are dimensioned and configured to allow only unidirectional relative movement of the first and second plate components.

In certain embodiments the distal end of at least of the first and second plate components has a lamina stabilizing flange such that the device can be positioned to be in contact with an anterior, posterior, superior and inferior surfaces of a divided lamina functioning to grip a cut edge of the lamina and aiding in positioning and securing the plate to the lamina. In certain preferred embodiments the lamina-stabilizing flange is in the form of a claw-like protrusion, while in other embodiments the lamina-stabilizing flange comprises a cuff that surrounds a portion of a transected lamina. In still other embodiments the lamina-stabilizing flange comprises a cup that completely encloses a portion of a transected lamina.

Certain embodiments of the bone fixation devices of the present invention have a bone graft-engaging portion configured to aid in the retention of an bone graft or allograft along a portion of the length of the graft when the graft is disposed within a surgically created gap a lamina. In certain embodiments the bone graft-engaging portion is a hollow recess disposed within a surface of the elongated fixation plate. In certain embodiments the bone graft-engaging portion in the form of a hollow recess disposed within a surface of either the first plate component, second plate component or both plate components. Such hollows or recesses can assume a variety of configurations and geometrical shapes including, but not limited to, round, ovoid, polygonal such as triangular or rectangular, and the like. In certain other embodiments the bone graft-engaging portion comprises deformable fingers or finger-like protrusions, which can be readily configured to engage the bone graft.

In certain embodiments the device the slidably coupled first and second plate components can be conveniently separated by utilizing forceps designed such that the gripping ends separate as the grasping ends are brought together.

The present invention also relates to methods of providing a desired distance between first and second cut bone ends produced during a laminoplasty procedure. A typical method comprises the steps of: (a) performing a laminoplasty procedure to produce a transected lamina; (b) providing a bone fixation device as herein described; (c) fixedly attaching the distal end of the first plate component to a lamina facet on one side of a transected lamina and fixedly attaching the distal end of the second plate component to a lamina facet on the side of the transected lamina opposite the side to which the distal end of the first component is fixedly attached; (d) slidably adjusting the first plate component and second plate component to produce a desired overall fixation plate length while separating the lamina at the transection to created an open space in the lamina; and (e) utilizing the locking means to fix the position of the first plate component and second plate component relative to one another. In certain embodiments such a method further comprises the step of securing a bone graft within the open space in the lamina.

### DETAILED DESCRIPTION OF THE INVENTION

The present invention describes embodiments of an expandable implantable plate that is used for stabilization of a lamina in a laminoplasty procedure wherein the plate offers controlled opening of a space within a transected lamina with integrated fixation. In preferred embodiments the plate is comprised of a first plate component slidably coupled to a second plate component. In a typical laminoplasty procedure a lamina is first completely transected near the junction of the lateral mass and then a notch, often also referred to as a hinge, is created on the contralateral side the lateral mass. In a laminoplasty procedure utilizing an expandable plate of the present invention the distal end of either the first plate component or second plate component is fixedly attached to the lamina on one side of the transection and the distal end of the other component is fixedly attached to the lamina on the opposing side of the transection. The first plate component and the second plate component are then slidably adjusted to a desired overall plate length to expand the lamina at the transection thereby creating an open space or gap. Finally, the two plate components are the locked relative to one another in the desired positions.

In typical embodiments, the devices have one or more bone screw receiving holes at the distal ends of the first plate component and the second plate component, which permits fixed attachment to lamina facets with standard bone screws. Such bone screw receiving holes can assume any suitable shape such as round or elongate slots.

The locking of the position of a first plate component relative a second plate component can be achieved by a variety of means including, but not limited to, bolts, set screws, clamps, clips and the like. In certain preferred embodiments, wherein a first plate component is slidably coupled and disposed within a pocket of a second plate component, locking is achieved by incorporation of a ratchet mechanism. Such a mechanism consists of tooth-like protrusions are set any acute angle of less than 90 degrees, preferably of less than 60 degrees and more preferably less than 45 degrees, along the surface of the first plate component and are skewed toward the opening of the second plate component pocket and the second plate component comprises a prawl for engaging the tooth-like protrusions of the first plate component, wherein the tooth-like protrusions and the prawl are dimensioned and configured to permit only unidirectional relative movement of the first and second plate components when the engaging flat (opposite face of teeth and prawl) surfaces of the are coplanar. The ratchet mechanism as herein described is intended to be exemplary and it is understood that variations of this and other ratchet mechanisms will be apparent to one skilled the art.

In certain other preferred embodiments the distal end of at least of the first and second plate components has a lamina stabilizing flange which can be positioned to be in contact with an anterior, posterior, superior and inferior surfaces of a divided lamina functioning to grip a cut edge of the lamina and aiding in positioning and securing the plate to the lamina. Suitable stabilizing flanges can be configured in a variety of forms including, but not limited to, a claw-like protrusion that engages an edge of a divided lamina, a cuff that substantially surrounds a portion of a divided lamina and a cup that completely encloses the first portion of the divided lamina. Suitable stabilizing flanges are those described in the published US Pat. Appl. US 2004/0030388 to Null et al., which is being included herein in its entirety by way of reference.

In certain laminoplasty procedures a bone or bone generating material of either a biologic or non-biologic nature is inserted into the gap. For use in such procedures embodiments of the expandable laminoplasty plates of the present invention are provided with a recess, a graft window or similar feature to assist in placement and/or retention of such bone generating material. A suitable configuration for such a bone graft retaining recesses or windows is disclosed in U.S. Pat. No. 6,635,087 to Angelucci et al., which is being included herein in its entirety by way of reference. Another suitable configuration for a bone graft or bone generating material retention is the configuration of the fixed-length as NewBridge™ Laminoplasty Fixation System available from Orthofix Spinal Implants, McKinney, Tex. USA. A list of appropriate bone or bone generating material for use in these embodiments includes, but is not limited to allografts such as fresh bone, freshly frozen bone, freeze-dried bone allograft (FDBA), demineralized freeze-dried bone allograft (DFDBA), demineralized bone matrix (DBM) containing bone morphogenetic protein (BMP), ceramics, calcium phosphates such as hydroxyapatite or tricalcium phosphate and the like.

In situ expansion of the embodiments of an expandable laminoplasty plate of the present invention during a laminoplasty procedure can be achieved by a variety of means including, but not limited to, manual separation of the slidably coupled first and second plate components and use of various surgical tools or instruments. In certain preferred embodiments separation of the slidably coupled first and second plate components is conveniently achieved by the use of forceps designed such that the gripping ends separate as the grasping ends are brought together.

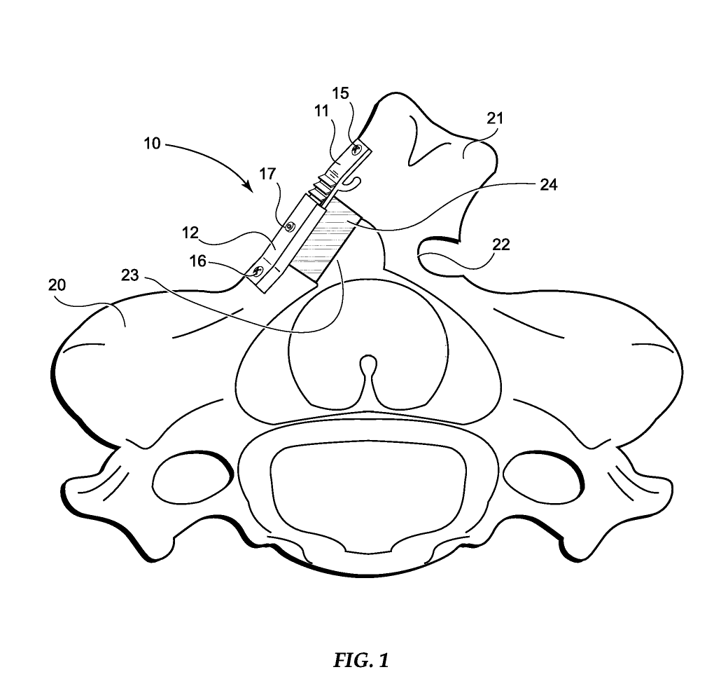

An embodiment of the invention attached to a lamina in the final position after a laminoplasty procedure is illustrated in FIG. 1 wherein a lamina 20 has been transected near the junction of the lateral mass 21 and a hinge 22 has been created on the contralateral side. The bone fixation device 10 comprises a second plate component 11 the proximal end of which is essentially a flat plate slidably coupled within a pocket formed within the proximal end of the first plate component 12, wherein the distal end of the second plate component 11 is fixedly attached to a facet of the lamina by a bone screw 15 while the distal end of the first plate component 12 is fixedly attached to a facet of the lamina by a bone screw 16 and the second plate component 11 is fixedly attached to a facet of the lamina by a bone screw 16. Since the second plate component 11 and first plate component 12 are slidably coupled, the length of the plate is conveniently adjusted to a desired length thereby expanding the gap 23 in the lamina 20 to a desired dimension. An optional locking means for fixing the relative positions of the second plate component 11 and first plate component 12 is depicted in FIG. 1 as setscrew 17. Also in the embodiment illustrated in FIG. 1 a bone allograft 24 has been inserted within the gap 23.

Another embodiment of the invention is illustrated in FIG. 2 wherein an expandable lamina fixation device 30 is depicted in a relatively closed or contracted position along with device-expanding forceps 40. The expandable fixation device 30 comprises and second plate component 31 slidably coupled and disposed within a pocket within a first plate component 32. In this embodiment the second plate component 31 comprises part of a ratchet mechanism in the form of tooth-like protrusions 35 set an acute angle such to be skewed toward the opening in the first plate component 32 and the first plate component 32 comprises a prawl 36 for engaging tooth-like protrusions 35, wherein the tooth-like ratchet components 35 and the prawl 36 are dimensioned and configured to allow only unidirectional relative movement of the first plate component 31 and the second plate component 32 when the engaging flat (opposite face of teeth and prawl) surfaces of the elements are coplanar. An additional feature of this embodiment is the claw-like protrusion 33 positioned near the distal end of first plate component 31 and which functions to grip a cut edge of a lamina and aids in positioning and securing the device 30 to a lamina. In FIG. 3 is illustrated the same expandable lamina fixation device depicted in FIG. 2, wherein the lamina fixation device 30 is in an expanded or open position along with plate-expanding forceps 40. This illustration clearly depicts the ratchet teeth 35 disposed along a surface of first plate component 31 as well as the prawl 36 disposed within the opening second plate component 32. FIG. 3 also clearly depicts bone screw receiving holes 37 and 38 at the distal ends of components 32 and 31 respectively wherein the holes are sized to accept standard bone screws 39. In use, the lengthening of the device 30 the positioning of the first plate component 31 and second plate component 32 relative to one another along the longitudinal axis effects distraction of a lamina (i.e. expanding the space in a lamina) to create a gap and further stabilizes the distracted lamina.

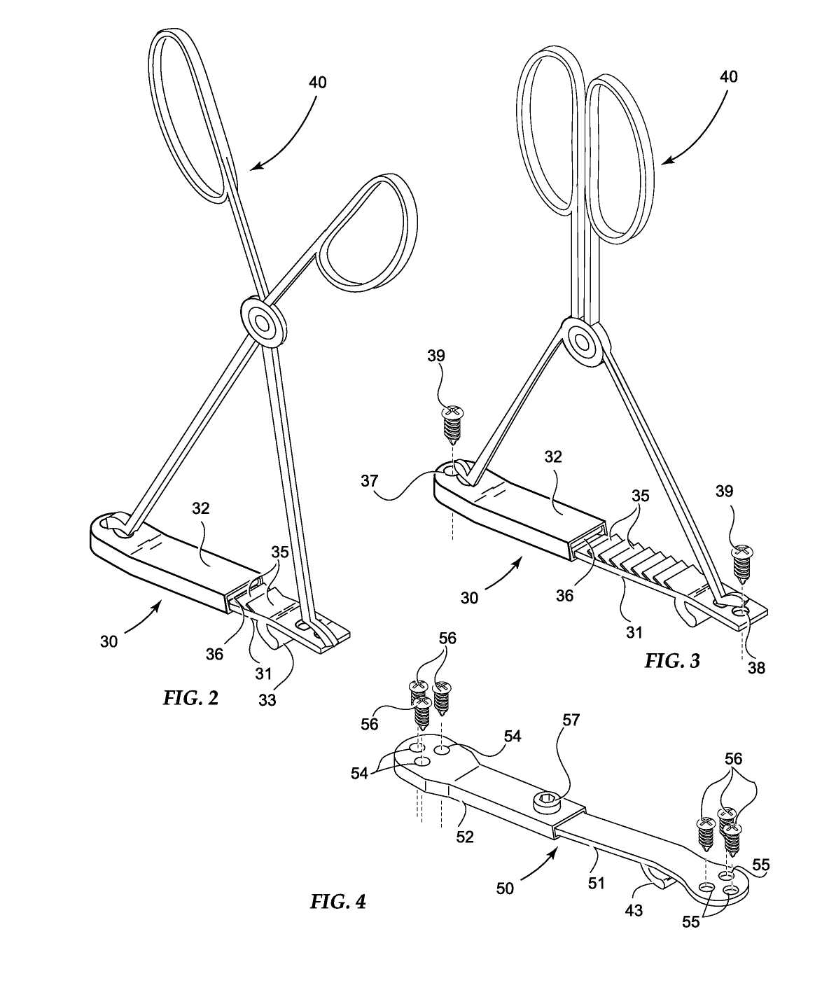

Another embodiment of the present invention is illustrated by FIG. 4 depicting an expandable bone plate 50 in an expanded or open position. The plate 50 comprises a first or inner plate component 51 slidably disposed within a second or outer plate component 52. The first plate component 51 further comprises a lamina stabilizing flange 53 in the form of a claw-like protrusion positioned near the distal end of inner component 51 and which functions to grip a cut edge of a lamina and aids in positioning and securing the bone plate 50 to the lamina. The expandable bone plate 50 also comprises screw-receiving holes 54 and 55 at the distal ends of components 52 and 51 respectively wherein the holes are sized to accept bone screws 56. The expandable bone plate 50 also comprises a set screw 57 that functions as a locking mechanism to secure the position of inner component 51 and outer component 52 relative to one another.

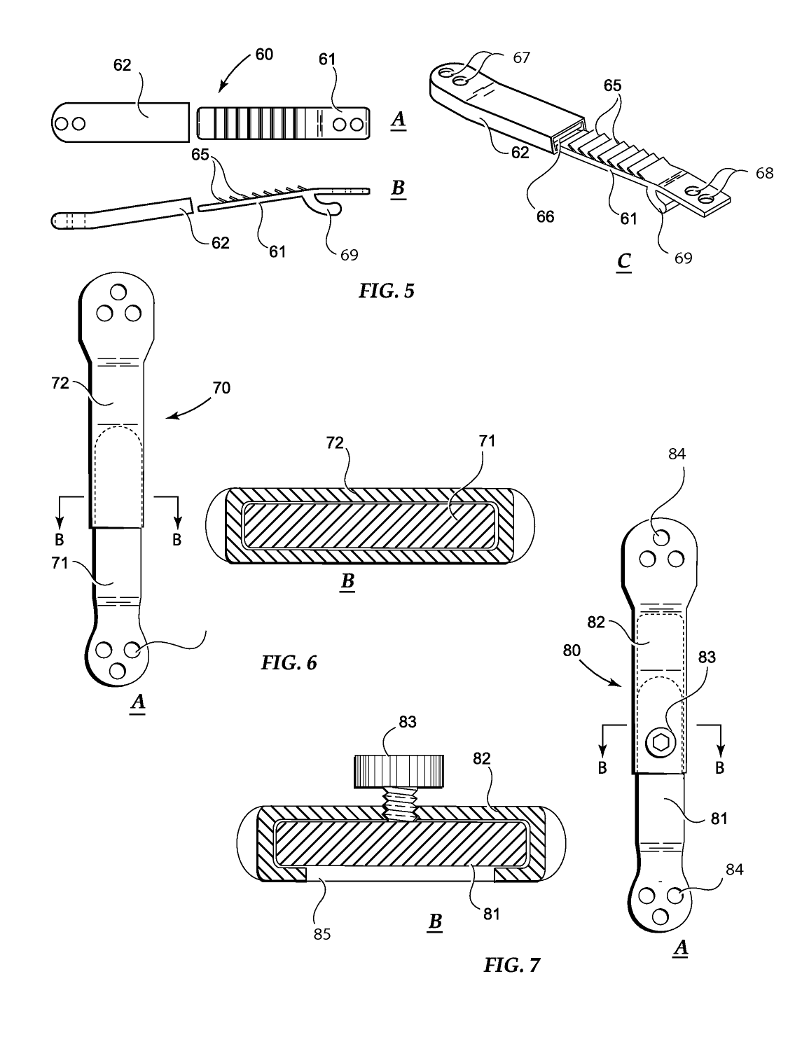

FIGS. 5A, 5B and 5C depict an expandable bone plate assembly 60 provided with a ratchet mechanism that allows the plate to be expanded (lengthened) along the longitudinal axis while preventing the plate from returning too a shortened position. FIG A is an orthogonal top view of a disengaged inner component 61 and outer component 62; FIG. 5b is an orthogonal side view of disengaged inner component 61 and outer component 62; and FIG. 5c is an isometric view of inner component 61 and outer component 62 when engaged. In this embodiment the inner component 61 comprises a series of linear ratchet components 65 in the form of tooth-like protrusions set an acute angle such that they are skewed toward a pocket-like opening the outer element 61 and the outer element 61 comprises a prawl 66 for engaging ratchet components 65, wherein the ratchet components 65 and the prawl 66 are dimensioned and configured to allow only unidirectional relative movement of the inner element 61 and the outer element 62 when the engaging flat (opposite face of teeth and prawl) surfaces of the elements are coplanar. Effectively when the engaging surfaces of the elements are coplanar which limits the plate to expansion of the length. However, in certain embodiments the ratchet mechanism is dimensioned and configured such that a ratcheted plate can be restored to a contracted or closed position by adjusting the orientation of the elements to one another. Another feature of the embodiment depicted in FIG. 5 is the lamina stabilizing flange 69 which can be positioned to be in contact with an anterior, posterior, superior and inferior surfaces of a divided lamina. Round screw-receiving holes 67 and 68 are also depicted.

In FIG. 6A is illustrated an orthogonal top view of an embodiment of an expandable bone plate 70 and in FIG. 6B a is illustrated cut sectional end view of the same expandable bone plate 70, wherein an first or inner component 71 is slidably disposed in pocket within second or outer component 72. The cut sectional view FIG. 6B clearly shows the inner component 71 disposed within a fully closed pocket or envelope in outer component 72. Round screw-receiving holes 73 are also depicted in FIG. 6A

In FIG. 7A is illustrated an orthogonal top view of an embodiment of an expandable bone plate 80 and in FIG. &B a is illustrated cut sectional end view of the same expandable bone plate 80 wherein an inner component 81 is slidably disposed within an outer component 82 and wherein the plate comprises a set-screw locking mechanism 83. The cut sectional view 7B clearly shows the inner component 81 disposed and maintained within a partially closed pocket or envelope in outer component 82 and wherein the inner component is locked in place with the outer component by the pressure exerted against the inner component 81 by the setscrew 83. Furthermore in this embodiment the partial opening 85 in the pocket outer component 82 provides a recess that is a suitable bone graft-engaging portion of the expandable bone plate 80. Round screw-receiving holes 84 are also depicted in FIG. 7A.

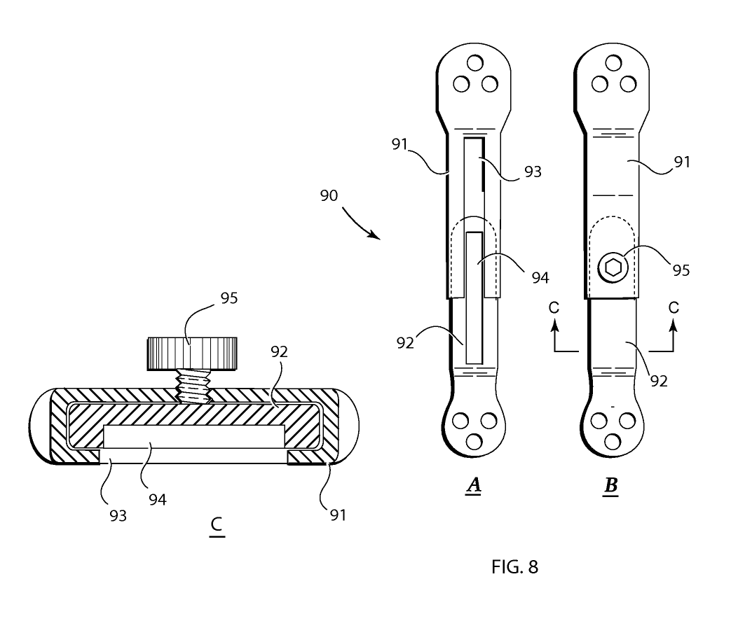

In FIG. 8A is illustrated an orthogonal bottom view of an embodiment of an expandable bone plate 90, in FIG. 8B an orthogonal top view of the expandable bone plate 90 and in FIG. 8B is illustrated cut sectional end view of the same expandable bone plate 90 wherein an inner component 92 is slidably disposed within an outer component 91 and wherein the plate comprises a set-screw locking mechanism 95. In this embodiment the inner component 92 has a rectangular recess 94 configured to provide a bone graft-engaging portion of the expandable bone plate 90 and wherein a partial opening 93 in the pocket of the outer component 91 forms an additional rectangular recess to act as bone graft-engaging feature.

It should be understood that certain features of the expandable bone plate fixation system configurations depicted in the accompanying figures are for illustrative purposes and that many variations are possible and in certain embodiments necessary. For example, there is no limitation with respect the angles formed between the ends of the plate and the major plate body which can be anywhere in the range of 0° to 90° and is chosen to conform to the geometry as dictated by the surgical procedure.

Materials useful for fabrication of embodiments of the implants of the present invention include any bio-compatible material having sufficient strength to maintain the open position of the divided lamina. Examples of suitable materials include, but are not limited to, titanium, titanium alloys, ceramics, composites, plastic composites, PEEK (polyetheretherketone) or PAEK (polyaryletherketone) as well as bioresorbable materials such as polylactides, polyglycolides and copolymers and blends thereof. The laminoplasty plates can also be constructed of a material that is radiolucent and/or bone growth inducing. In certain specific embodiments, a particularly suitable material is the titanium alloy designated as CP Ti grade 2 alloy.

Other embodiments of the devices and methods of the present invention will become apparent to those skilled in the art based on the description and drawings of the embodiments presented herein and the present invention is in no way limited to these embodiments.

We claim:

1. A bone fixation device for the lamina of the spine after laminoplasty comprising:
an elongated fixation plate having adjustable length comprising a first plate component having a distal end, a proximal end and a longitudinal axis extending there between; said first plate component being slidebly coupled to a second plate component having a distal end, a proximal end and a longitudinal axis extending there between; wherein
the distal end of the first plate component is configured to be fixedly attachable to a lamina facet on one side of a transected lamina and wherein the distal end of the second plate component is configured to be fixedly attachable to a lamina facet on the side of the transected lamina opposite the side to which the distal end of the first component is fixedly attachable; wherein
the first plate component and second plate component can be slidably adjusted to produce a desired overall fixation plate length while the distal ends of the first and second plate components are each fixedly attached to opposing facets of the transected lamina and the two plate components are the locked relative to one another in the desired positions; and
a locking means to secure the position of the first plate component and second plate component relative to one another.

2. The bone fixation device of claim 1 wherein the distal end of the first plate component and the distal end of the second plate component each comprise one or more bone screw receiving holes configured to permit fixed attachment to lamina facets with bone screws.

3. The bone fixation device of claim 1 wherein said one or more bone screw receiving holes is an elongated slot.

4. The bone fixation device of claim 1 wherein the locking means comprises a setscrew.

5. The bone fixation device of claim 1 wherein the locking means comprises a ratchet mechanism.

6. The bone fixation device of claim 1 wherein the proximal end of the first plate component is slidably coupled within a pocket in the proximal end of the second plate component.

7. The bone fixation device of claim 5 wherein the locking means comprises a ratchet mechanism comprising a series of tooth-like protrusions set an angle less of than 90 degrees along a surface of the first plate component and skewed toward the opening of the second plate component pocket and wherein the second plate component comprises a prawl for engaging the tooth-like protrusions of the first plate component, wherein the tooth-like protrusions and the prawl are dimensioned and configured to allow only unidirectional relative movement of the first and second plate components.

8. The bone fixation device of claim 1 wherein the distal end of at least of the first and second plate components has a lamina stabilizing flange such that the device can be positioned to be in contact with an anterior, posterior, superior and inferior surfaces of a divided lamina functioning to grip a cut edge of the lamina and aiding in positioning and securing the plate to the lamina.

9. The bone fixation device of claim 1 wherein the lamina stabilizing flange comprises a claw-like protrusion.

10. The bone fixation device of claim 1 wherein the lamina stabilizing flange comprises a cuff that surrounds a portion of a transected lamina.

11. The bone fixation device of claim 1 wherein the lamina stabilizing flange comprises a cup that completely encloses a portion of a transected lamina.

12. The bone fixation device of claim 1 further comprising a bone graft-engaging portion configured to aid in retention of an allograft along a portion of the length of the allograft when the allograft is disposed within a surgically created gap a lamina.

13. The bone fixation device of claim 12 wherein the bone graft-engaging portion comprises a hollow recess disposed within a surface of the elongated fixation plate.

14. The bone fixation device of claim 12 wherein the hollow recess is rectangular in shape.

15. The bone fixation device of claim 12 wherein the bone graft-engaging portion comprises deformable fingers configured to engage the bone graft.

16. The bone fixation device of claim 6 further comprising a bone graft-engaging portion in the form of a hollow recess is disposed within a surface of the second plate component.

17. The bone fixation device of claim 16 further comprising a bone graft-engaging portion in the form of a hollow recess is disposed within a surface of the first plate component.

18. The bone fixation device of claim 1 wherein at least a portion of the device is fabricated from a material comprising titanium or a titanium alloy.

19. The bone fixation device of claim 1 wherein at least a portion of the device is fabricated from a material comprising a polyetheretherketone or a polyaryletherketone.

20. A bone fixation kit for use in the lamina of the spine after laminoplasty comprising a device of claim 1 and forceps for the separation of the slidably coupled first and second plate components wherein the forceps comprise gripping ends that separate as grasping ends are brought together.

21. A method of providing a desired distance between first and second cut bone ends produced during a laminoplasty procedure, comprising the steps of:
(a) performing a laminoplasty procedure to produce a transected lamina;
(b) providing a bone fixation device of claim 1;
(c) fixedly attaching the distal end of the first plate component to a lamina facet on one side of a transected lamina and fixedly attaching the distal end of the second plate component to a lamina facet on the side of the transected lamina opposite the side to which the distal end of the first component is fixedly attached;
(d) slidably adjusting the first plate component and second plate component to produce a desired overall fixation plate length while separating the lamina at the transection to created an open space in the lamina; and
(e) utilizing the locking means to fix the position of the first plate component and second plate component relative to one another.

22. The method of claim 20 further comprising the step of securing a bone graft within the open space in the lamina.
